(19) United States
(12) Patent Application Publication
(10) Pub. No.: US 2017/0211032 A1
(43) Pub. Date: Jul. 27, 2017

(54) TEMPERATURE REGULATING CONTAINER

(71) Applicant: HITACHI HIGH-TECHNOLOGIES CORPORATION, Tokyo (JP)

(72) Inventors: Akira MASUYA, Tokyo (JP); Muneo MAESHIMA, Tokyo (JP); Yasunori SHOJI, Tokyo (JP)

(73) Assignee: HITACHI HIGH-TECHNOLOGIES CORPORATION, Tokyo (JP)

(21) Appl. No.: 15/326,201

(22) PCT Filed: Jun. 17, 2015
(86) PCT No.: PCT/JP2015/067387

§ 371 Date: Jan. 13, 2017

(30) Foreign Application Priority Data
Jul. 14, 2014 (JP) 2014-143802

(51) Int. Cl. C12M 1/02; C12M 1/32; C12M 1/00

(52) U.S. Cl. CPC C12M 41/22; C12M 23/38; C12M 23/12

(57) ABSTRACT

A temperature regulating container handles multiple samples which can be optically measured in real time while regulating the temperature of the samples. The samples can be moved at high speed by, for example, shaking without requiring a complicated mechanism. A cell culture container is a combination of three kinds of members: a temperature regulating liquid-holding member, a sample-holding member, and a lid. The cell culture container ensures that multiple samples are contacted with a temperature regulating liquid so that the temperature of the samples can be maintained at a uniform temperature, or quickly increased. The lid contacts the surface of the samples, preventing the samples from drying and contamination and, preventing dew formation on an optical path for performing optical measurement. The distance the light passes through the samples can be held constant to thereby assure accurate optical measurement; and sample containers can be easily moved.

### TECHNICAL FIELD

The present invention relates to a cell culture container which can stably hold a liquid whose temperature is regulated between multiple sample containers.

### BACKGROUND ART

As for cell culture, temperature management is one of the most important technical issues. In order to quickly and stably apply a culture temperature to cells, a temperature-controlled liquid is generally brought into contact with a sample container for culturing the cells. Advantageous effects of using the liquid for temperature regulating mainly include the following two points. (1) The liquid is more likely to transfer heat, compared to gas. (2) The liquid is deformed, and comes into close contact with an outer surface of the sample container. Accordingly, a contact area thereof can be maximized. Owing to these advantageous effects, heat is easily transferred to the sample container itself and a sample inside the container.

According to PTL 1, the liquid is injected to a portion between a heat block serving as a heat source and the sample container, thereby obtaining the advantageous effects (1) and (2).

Here, in order to monitor a culture process of the cells, optical measurement and culture are concurrently performed in many cases. For example, time-lapse imaging is performed in order to observe a differentiation process of the cells. According to PTL 1, if the sample container is detached from the heat block during optical measurement, an optical measurement surface is brought into a state where the liquid adheres to the optical measurement surface. Therefore, in order to perform the optical measurement, it is necessary to remove or clean the liquid on the optical measurement surface. However, if the optical measurement surface is frequently cleaned, there is a possibility that the optical measurement surface may be scratched.

According to PTL 2, in order to observe a state of the sample inside the sample container, the sample container is not separated from the temperature-regulated liquid, and the optical measurement is performed through the liquid.

Here, in order to perform the optical measurement through the temperature-regulated liquid, the temperature-regulated liquid needs to maintain a stable and high transmittance rate. In a state where the temperature-regulated liquid is released to the atmosphere in order to dip the sample container into the temperature-regulated liquid, the temperature-regulated liquid is held inside a device for a long time. Accordingly, due to contamination such as bacteria in the air, the temperature-regulated liquid becomes turbid, and the transmittance rate is changed. According to PTL 2, in order to prevent the transmittance rate from being changed due to the contamination, there is provided a device for sterilizing the contaminating bacteria. In addition, the sample container is dipped into the temperature-regulated liquid. Accordingly, if the sample container is moved or shaken fast, the temperature-regulated liquid bubbles up. Consequently, the temperature-regulated liquid cannot be stably held inside the device. An operation of uniformly mixing the sample by shaking the sample container is one of important operations in performing the cell culture.

### CITATION LIST

### Patent Literature

PTL 1: JP-A-2004-294130

PTL 2: JP-A-2013-134141

### SUMMARY OF INVENTION

### Technical Problem

In a method of removing a liquid on an optical measurement surface or cleaning the optical measurement surface, if the liquid on the optical measurement surface of a sample is quickly and automatically removed or cleaned so as not to affect optical measurement, a device requires a complicated configuration, and manufacturing cost of the device increases. Accordingly, it is difficult to perform the optical measurement in real time. According to a method of performing the optical measurement through a temperature-regulated liquid, in order to maintain a transmittance rate of the temperature-regulated liquid, it is necessary to provide a disinfection device of the temperature-regulated liquid, as a countermeasure against contamination such as bacteria, thereby resulting in mechanically complicated configuration. In addition, if a sample container is dipped into the temperature-regulated liquid, or if the sample container is moved or shaken fast, the temperature-regulated liquid cannot be stably held inside the device.

A problem to be solved is to provide a temperature regulating container in which each state of multiple samples can be optically measured in real time while each temperature of the multiple samples is regulated, and in which the sample can be shaken or moved fast without requiring a complicated mechanism.

### Solution to Problem

A temperature regulating container according to the present invention includes a temperature regulating liquid container that accommodates a temperature regulating liquid, a sample-holding member that can be accommodated inside the temperature regulating liquid container, and that has multiple recess portions for holding a sample, and a lid member that has multiple projection portions for putting a lid on each recess portion.

### Advantageous Effects of Invention

Each temperature of multiple samples can be uniformly regulated at any desired timing. Each state of the samples can be optically measured while the temperature is regulated. A sample container can be moved or shaken fast.

### BRIEF DESCRIPTION OF DRAWINGS

FIG. 1 is a perspective view illustrating characteristics of a cell culture container according to Embodiment 1.

FIG. 2 is a perspective view illustrating a sample setting method of the cell culture container according to Embodiment 1.

FIG. 3 is a perspective view illustrating a sample observation method of the cell culture container according to Embodiment 1.

FIG. 4 is a perspective view illustrating characteristics of a cell culture container according to Embodiment 2.

FIG. 5 is a perspective view illustrating a sample setting method of the cell culture container according to Embodiment 2.

FIG. 6 is a perspective view illustrating a sample observation method of the cell culture container according to Embodiment 2.

### DESCRIPTION OF EMBODIMENTS

The present invention provides a container which can hold a temperature regulating liquid around a sample container by interposing the temperature regulating liquid between a container for accommodating multiple samples and the other member. In addition, the present invention provides an introduction method of the sample and the temperature regulating liquid.

In addition, the present invention provides a container which has a portion for accommodating multiple samples and a portion for accommodating the temperature regulating liquid, in which one or more holes for a temperature regulating liquid inlet and an air hole are disposed in the portion for accommodating the temperature regulating liquid, and which can inject the temperature regulating liquid into the portion for accommodating the temperature regulating liquid through the inlet. In addition, the present invention provides an introduction method of the sample and the temperature regulating liquid.

Furthermore, the present invention provides a lid which removes an air layer on an optical path for optical measurement so as not to cause dew formed due to a temperature difference between the external air and the inside of the sample container, which can maintain a constant distance in which light passes through the inside of the sample, and which has a structure partially dipped into the sample.

A sample to be measured may be a chemical reagent in addition to a cell suspension.

Hereinafter, with regard to preferred embodiments for embodying the present invention, two embodiments will be described with reference to the drawings. In each embodiment, the following items will be described.

### 1. Outline of Each Member

### 2. Sample Setting Method

### 3. Incubation and Observation Method

The embodiments described herein are merely examples of representative embodiments of the present invention. The embodiments do not allow the scope of the present invention to be narrowly interpreted.

### Embodiment 1

### 1. Outline of Each Member

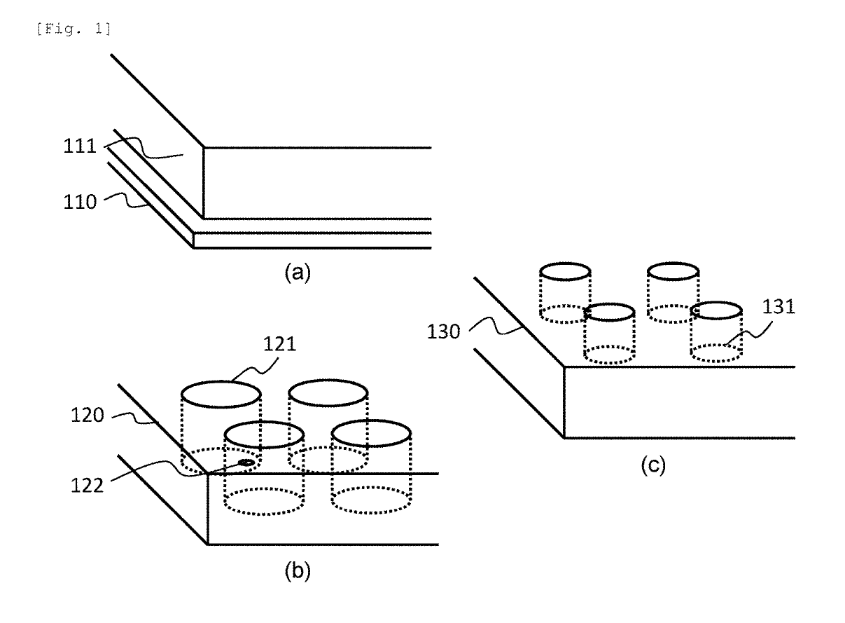

FIG. 1 is a perspective view illustrating a configuration and characteristics of a cell culture container according to the present invention.

The cell culture container is configured to include three members of a temperature regulating liquid-holding member 110, a sample-holding member 120, and a lid 130.

The temperature regulating liquid-holding member 110 illustrated in FIG. 1(a) has a structure surrounded by an edge having a temperature regulating liquid-holding portion 111 which is one partitioned region for holding a temperature regulating liquid. A size and a shape of the overall temperature regulating liquid-holding portion 111 are aligned with an outer edge of the sample-holding member 120 illustrated in FIG. 1(b). In this manner, a structure is employed in which the temperature regulating liquid is less likely to be scattered.

The sample-holding member 120 illustrated in FIG. 1(b) has a structure provided with multiple recessed sample-holding portions 121 which can dispense and hold the sample from above in a lid-shaped top plate. A cross-sectional shape of the sample-holding portion 121 may be circular or square. It is desirable that a bottom surface of the sample-holding portion 121 is flat and smooth. A size and a shape of the overall sample-holding member 120 are aligned with the temperature regulating liquid-holding portion 111 illustrated in FIG. 1(a). In this manner, the sample-holding member 120 is shaped so that the temperature regulating liquid is less likely to be scattered. In addition, the top plate of the sample-holding member 120 has one or more air holes 122 serving as through-holes. The air hole 122 is provided with an air exchange function when the temperature regulating liquid-holding member 110 and the sample-holding member 120 are combined with each other, and a function to maintain high humidity so as to prevent the sample from being dried. The air hole 122 may be covered with a waterproof moisture-permeable material so that the temperature regulating liquid is not scattered.

The lid 130 illustrated in FIG. 1(c) has a structure having multiple recessed sample contact portions 131 in the top plate having a normal lid shape. A size and a shape of the overall lid 130 are aligned with the sample-holding member 120 illustrated in FIG. 1(b). The number of the sample contact portions 131 is the same as the number of the sample-holding portions 121 illustrated in FIG. 1(b), and the sample contact portion 131 and the sample-holding portion 121 are disposed at the same position. A cross-sectional shape of the sample contact portion 131 is smaller than a cross-sectional shape of the sample-holding portion 121. The sample contact portion 131 is accommodated inside the sample-holding portion 121. A depth of the sample contact portion 131 is set to a depth which allows contact with the surface of the sample when the temperature regulating liquid-holding member 110, the sample-holding member 120, and the lid 130 are combined with each other.

The temperature regulating liquid-holding member 110, the sample-holding member 120, and the lid 130 employ a transparent member for a portion through which light passes during microscopic observation or optical density measurement or for all members. 2. Sample Setting Method

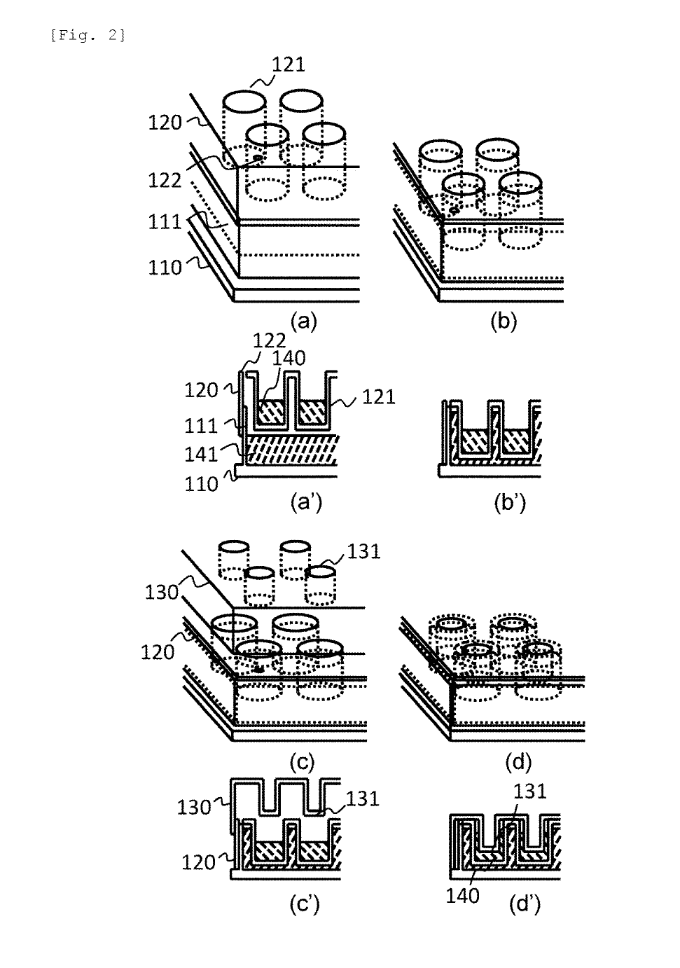

FIG. 2 schematically illustrates a sample setting method of the cell culture container.

As illustrated in a three-dimensional view (a) in FIG. 2 and a sectional view (a′) thereof, a sample 140 is put in the sample-holding portion 121 of the sample-holding member 120. Thereafter, a temperature-regulated temperature regulating liquid 141 which raises the temperature of the sample 140 up to a target temperature is dispensed to the temperature regulating liquid-holding portion 111. It is desirable to use a fluid with large specific heat such as water for the temperature regulating liquid 141.

As illustrated in a three-dimensional view (b) in FIG. 2 and a sectional view (b′) thereof, the sample-holding member 120 and the temperature regulating liquid-holding member 110 are caused to overlap each other, and the temperature regulating liquid 141 is brought into contact with the sample-holding portion 121. Here, the air is discharged from the air hole 122. In this manner, the temperature regulating liquid 141 is introduced into and held in a portion between the sample-holding portions 121.

As illustrated in a three-dimensional view (c) in FIG. 2 and a sectional view (c′) thereof, the lid 130 is put thereon from above the sample-holding member 120. Here, in a case where the air is exchanged under a culture condition of the sample 140, the sample-holding member 120 and the lid 130 are set to have a shape which is not sealed. In a case where the air is not exchanged, the sample-holding member 120 and the lid 130 are set to have a shape which is sealed.

As illustrated in a three-dimensional view (d) in FIG. 2 and a sectional view (d′) thereof, a bottom surface of the sample contact portion 131 is brought into contact with a surface of the sample 140.

In this way, sample setting is completed.

### 3. Incubation and Observation Method

With regard to an incubation method, in the sample container in which three members of the sample-holding member 120 having the sample 140 introduced thereto in FIG. 2, the temperature regulating liquid-holding member 110 having the temperature regulating liquid 141 introduced thereto, and the lid 130 are combined with each other, the temperature of the sample container is allowed to reach the target temperature by the temperature regulating liquid 141. In order to hold the temperature, the sample container is stored in an air thermostat which holds the temperature to be the same as the target temperature. In this way, the sample container is prevented from losing heat. In addition, in a case where the sample is to be mixed, the sample container is shaken.

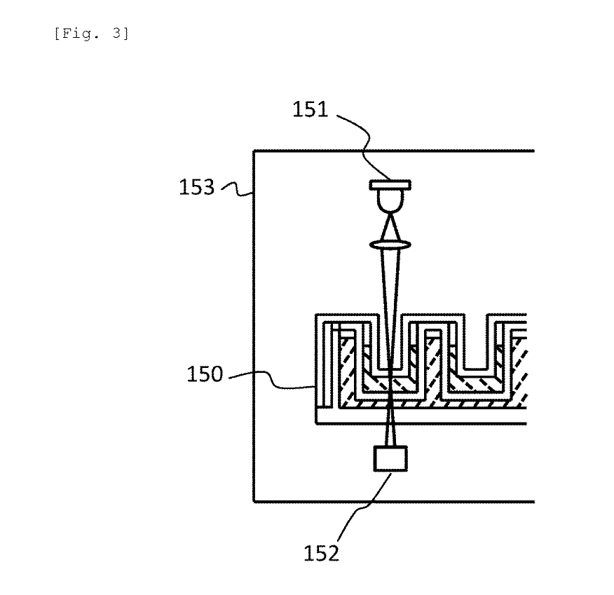

With regard to an observation method, as illustrated in FIG. 3, light emitted from a light source 151 in the thermostat 153 holding the temperature is condensed by a lens. The light is transmitted through the sample inside the sample container 150 used for incubation. The light is received by a photodetector 152. An observation value of optical density or turbidity of the sample is acquired from a signal obtained from the photodetector 152. In order to observe a culture state, the measurement is performed at any desired time interval such as one hour interval, thereby observing how cells proliferate. In addition, instead of the photodetector 152, an imaging element and an objective lens are disposed, and microscopic observation is performed on the sample. It is also possible to observe a proliferative process of the cells by comparing the observation images.

According to the present embodiment, even if a temperature environment is changed when the sample container is detached, dew is not formed on an optical path. In addition, it is possible to maintain a constant distance in which light passes through the inside of the sample, and it is possible to maintain a state where the temperature of the sample is less changed.

### Embodiment 2

### 1. Outline of Each Member

FIG. 3 is a perspective view illustrating a configuration and characteristics of a cell culture container according to the present invention.

The cell culture container is configured to include two members of a sample-holding member 210 and a lid 220.

The sample-holding member 210 illustrated in FIG. 3(a) has a structure provided with multiple sample-holding portions 211 having a recessed shape which the sample enters on an upper surface of a hollow box. A cross-sectional shape of the sample-holding portion 211 may be circular or square. It is desirable that the bottom surface of the sample-holding portion 211 is flat and smooth. A depth of the bottom surface of the sample-holding portion 211 may be equal to that of the bottom surface of the sample-holding member 210. Alternatively, a space may be disposed between the bottom surface of the sample-holding portion 211 and the bottom surface of the sample-holding member 210. An outer space of the sample-holding portion 211 inside the sample-holding member 210 serves as temperature regulating liquid-holding portions 212. In order to uniformly and quickly raise the temperature of the sample inside the multiple sample-holding portions 211, it is desirable to install multiple temperature regulating liquid inlets 214 on the upper surface or the side surface of the sample-holding member so that the temperature regulating liquid can be introduced fast. The temperature regulating liquid inlet 214 may be provided with a structure having a check valve so that the liquid does not leak when the liquid is injected. An air hole 213 is installed on the upper surface of the sample-holding member, and is provided with an air exchange function when the temperature regulating liquid is introduced, and a function to maintain high humidity so as to prevent the sample from being dried. The air hole 213 may be covered with a film made of a waterproof moisture-permeable material so that the temperature regulating liquid is not scattered.

The lid 220 illustrated in FIG. 3(b) has a structure having multiple recessed sample contact portions 221 in the top plate having a normal lid shape. A size and a shape of the overall lid 220 are aligned with the sample-holding member 210 illustrated in FIG. 3(a). The number of the sample contact portions 221 is the same as the number of the sample-holding portions 211 illustrated in FIG. 3(a), and the sample contact portion 221 and the sample-holding portion 211 are disposed at the same position. A cross-sectional shape of the sample contact portion 221 is smaller than a cross-sectional shape of the sample-holding portion 211. The sample contact portion 221 is accommodated inside the sample-holding portion 211. A depth of the sample contact portion 221 is set to a depth which allows contact with the surface of the sample when the sample-holding member 210 and the lid 220 are combined with each other.

The sample-holding member 210 and the lid 220 employ a transparent member for a portion through which light passes during microscopic observation or optical density measurement or for all members.

### 2. Sample Setting Method

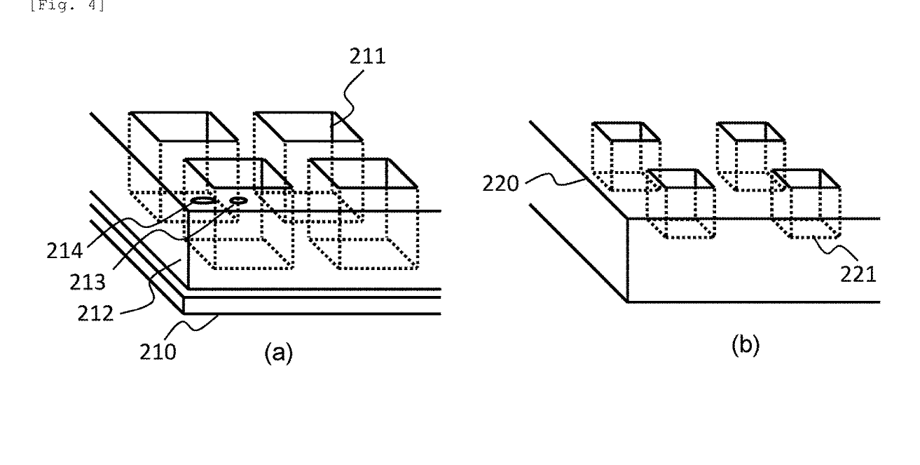

FIG. 4 schematically illustrates a sample setting method of the cell culture container.

As illustrated in a three-dimensional view (a) in FIG. 4 and a sectional view (a′) thereof, a sample 230 is put in the sample-holding portion 211 of the sample-holding member 210.

As illustrated in a three-dimensional view (b) in FIG. 4 and a sectional view (b′) thereof, a temperature-regulated temperature regulating liquid 231 which raises the temperature of the sample 230 up to a target temperature is introduced to the temperature regulating liquid-holding portion 212 through the temperature regulating liquid inlet 214. It is desirable to use a fluid with large specific heat such as water for the temperature regulating liquid 231.

As illustrated in a three-dimensional view (c) in FIG. 4 and a sectional view (c′) thereof, the lid 220 is put thereon from above the sample-holding member 210. Here, in a case where the air is exchanged under a culture condition of the sample 230, the sample-holding member 210 and the lid 220 are set to have a shape which is not sealed. In a case where the air is not exchanged, the sample-holding member 210 and the lid 220 are set to have a shape which is sealed.

As illustrated in a three-dimensional view (d) in FIG. 2 and a sectional view (d′) thereof, the bottom surface of the sample contact portion 221 is brought into contact with the surface of the sample 230.

In this way, sample setting is completed.

### 3. Incubation and Observation Method

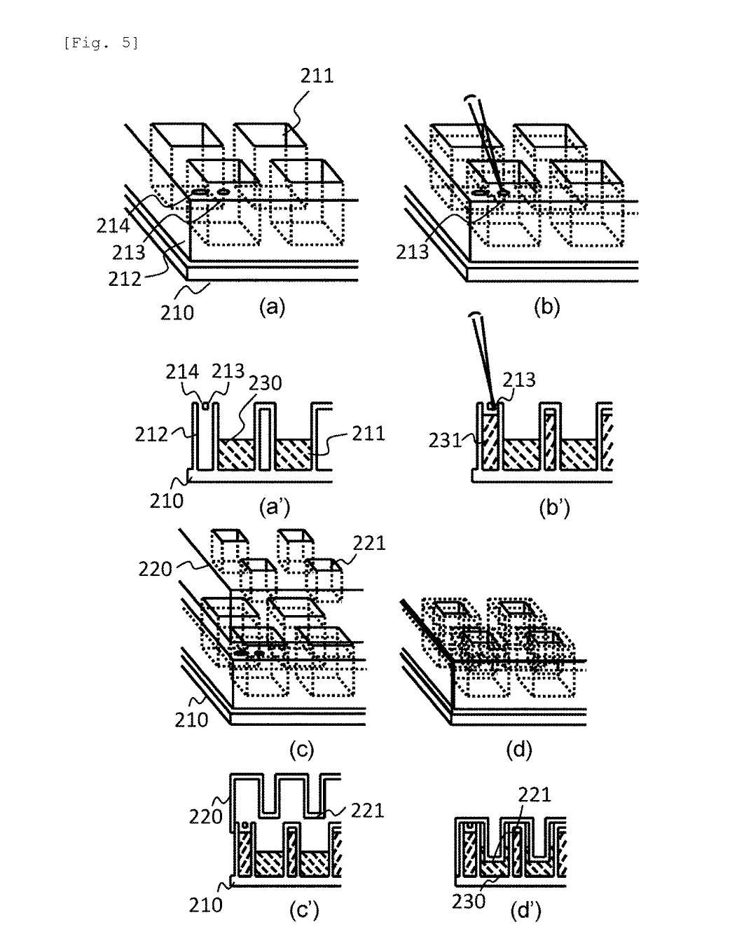

With regard to an incubation method, in the sample container in which the sample-holding member 120 having the sample 230 introduced thereto in FIG. 5 and the lid 220 are combined with each other, the temperature of the sample container is allowed to reach the target temperature by the temperature regulating liquid 231. In order to hold the temperature, the sample container is stored in an air thermostat which holds the temperature to be the same as the target temperature. In this way, the sample container is prevented from losing heat. In addition, in a case where the sample is to be mixed, the sample container is shaken.

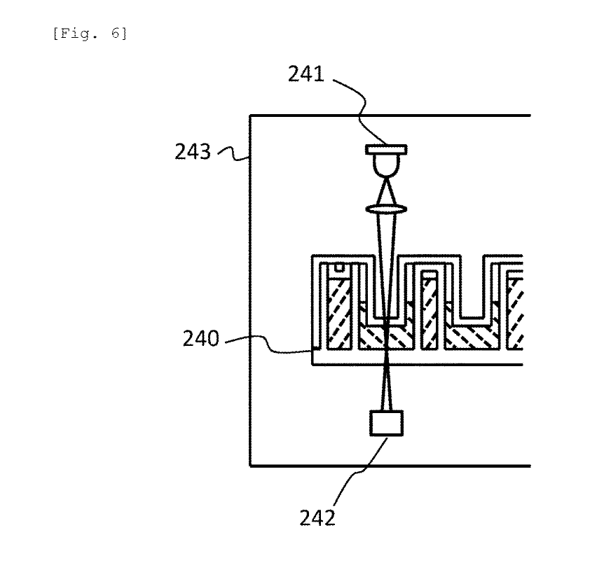

With regard to an observation method, as illustrated in FIG. 6, light emitted from a light source 241 in a thermostat 243 holding the temperature is condensed by a lens. The light is transmitted through the sample inside the sample container 240 used for incubation. The light is received by a photodetector 242. An observation value of optical density or turbidity of the sample is acquired from a signal obtained from the photodetector. In order to observe a culture state, the measurement is performed at any desired time interval such as one hour interval, thereby observing how cells proliferate. In addition, instead of the photodetector 242, an imaging element and an objective lens are disposed, and microscopic observation is performed on the sample. It is also possible to observe a proliferative process of the cells by comparing the observation images.

### REFERENCE SIGNS LIST

110 temperature regulating liquid-holding member
111 temperature regulating liquid-holding portion
120 sample-holding member
121 sample-holding portion
122 air hole
130 lid
131 sample contact portion
140 sample
141 temperature regulating liquid
150 sample container
151 light source
152 photodetector
153 thermostat
210 sample-holding member
211 sample-holding portion
212 temperature regulating liquid-holding portion
213 temperature regulating liquid inlet
214 air hole
220 lid
221 sample contact portion
230 sample
231 temperature regulating liquid
240 sample container
241 light source
242 photodetector
243 thermostat

1. A temperature regulating container comprising:
a temperature regulating liquid container that accommodates a temperature regulating liquid;
a sample-holding member that can be accommodated inside the temperature regulating liquid container, and that has multiple recess portions for holding a sample; and
a lid member that has multiple projection portions for putting a lid on each recess portion,
wherein the lid member has a sample contact portion.

2. The temperature regulating container according to claim 1,
wherein the temperature regulating liquid container, the sample-holding member, and the lid member can be fixed to each other by overlapping each other in this order.

3. The temperature regulating container according to claim 2,
wherein when the temperature regulating liquid container, the sample-holding member, and the lid member overlap each other, at least a portion of the projection portion is accommodated inside the recess portion.

4. The temperature regulating container according to claim 1,
wherein the sample-holding member has at least one through-hole.

5. The temperature regulating container according to claim 4,
wherein the through-hole is covered with a waterproof moisture-permeable material.

6. The temperature regulating container according to claim 1,
wherein at least a portion of the temperature regulating liquid container, the sample-holding member, and the lid member is formed of a transparent member for optical observation.

7. The temperature regulating container according to claim 2,
wherein in a state where the temperature regulating liquid container, the sample-holding member, and the lid member overlap each other, the temperature regulating liquid is accommodated in a closed space.

8. A temperature regulating container comprising:
a sample-holding member that has multiple recess portions for holding a sample; and
a lid member that has multiple projection portions for putting a lid on each recess,
wherein the sample-holding member includes an accommodation portion that accommodates the temperature regulating liquid around each recess portion, and
wherein the lid member has a sample contact portion.

9. The temperature regulating container according to claim 8,
wherein the sample-holding member and the lid member can be fixed to each other by overlapping each other in this order.

10. The temperature regulating container according to claim 9,
wherein when the sample-holding member and the lid member overlap each other, at least a portion of the projection portion is accommodated inside the recess portion.

11. The temperature regulating container according to claim 8,
wherein the sample-holding member has at least one through-hole.

12. The temperature regulating container according to claim 11,
wherein the through-hole is covered with a waterproof moisture-permeable material.

13. The temperature regulating container according to claim 8,
wherein the sample-holding member includes a liquid inlet for introducing the temperature regulating liquid.

14. The temperature regulating container according to claim 13,
wherein the liquid inlet includes a check valve.

15. The temperature regulating container according to claim 8,
wherein at least a portion of the sample-holding member and the lid member is formed of a transparent member for optical observation.

16. The temperature regulating container according to claim 9,
wherein in a state where the sample-holding member and the lid member overlap each other, the temperature regulating liquid is accommodated in a closed space.

17. The temperature regulating container according to claim 1,
wherein the sample contact portion is disposed in a distal end of the projection portion of the lid member, and comes into contact with a surface of the sample inside the recess portion of the sample-holding member.

18. The temperature regulating container according to claim 8,
wherein the sample contact portion is disposed in a distal end of the projection portion of the lid member, and comes into contact with a surface of the sample inside the recess portion of the sample-holding member.
